SEQUENCE LISTING (16 sequences)

SEQ ID NO: 1 - RNA, 11 nt, Artificial Sequence
  synthetic
gacgugcgaag

SEQ ID NO: 2 - RNA, 11 nt, Artificial Sequence
  synthetic
cuucgcacguc

SEQ ID NO: 3 - DNA, 11 bp, Artificial Sequence
  synthetic
gacgtgcgaag

SEQ ID NO: 4 - DNA, 11 bp, Artificial Sequence
  synthetic
cttcgcacgtc

SEQ ID NO: 5 - RNA, 22 nt, Artificial Sequence
  synthetic
gacgugcgaaggacgugcgaag

SEQ ID NO: 6 - RNA, 22 nt, Artificial Sequence
  synthetic
cuucgcacguccuucgcacguc

SEQ ID NO: 7 - DNA, 22 bp, Artificial Sequence
  synthetic
gacgtgcgaaggacgtgcgaag

SEQ ID NO: 8 - DNA, 22 bp, Artificial Sequence
  synthetic
cttcgcacgtccttcgcacgtc

SEQ ID NO: 9 - RNA, 14 nt, Artificial Sequence
  synthetic
cgugcgacgugcga

SEQ ID NO: 10 - RNA, 21 nt, Artificial Sequence
  synthetic
cgugcgacgugcgacgugcga

SEQ ID NO: 11 - RNA, 14 nt, Artificial Sequence
  synthetic
ucgcacgucgcacg

SEQ ID NO: 12 - RNA, 21 nt, Artificial Sequence
  synthetic
ucgcacgucgcacgucgcacg

SEQ ID NO: 13 - DNA, 14 bp, Artificial Sequence
  synthetic
cgtgcgacgtgcga

SEQ ID NO: 14 - DNA, 21 bp, Artificial Sequence
  synthetic
cgtgcgacgtgcgacgtgcga

SEQ ID NO: 15 - DNA, 14 bp, Artificial Sequence
  synthetic
tcgcacgtcgcacg

SEQ ID NO: 16 - DNA, 21 bp, Artificial Sequence
  synthetic
tcgcacgtcgcacgtcgcacg

(19) United States
(12) Patent Application Publication
(10) Pub. No.: US 2016/0201118 A1
(43) Pub. Date: Jul. 14, 2016

(54) NUCLEIC ACID CALIBRATION STANDARDS

(71) Applicants: Paul F. AGRIS, Albany, NY (US); Ken HALVORSEN, Glenmont, NY (US)

(72) Inventors: Paul F. AGRIS, Albany, NY (US); Ken HALVORSEN, Glenmont, NY (US)

(21) Appl. No.: 14/854,129

(22) Filed: Sep. 15, 2015

Related U.S. Application Data
(63) Continuation of application No. PCT/US2014/030416, filed on Mar. 17, 2014.
(60) Provisional application No. 61/789,409, filed on Mar. 15, 2013.

(51) Int. Cl. C12Q 1/68

(52) U.S. Cl. CPC C12Q 1/6825

(57) ABSTRACT

The invention relates to synthetic nucleic acids and in particular, complementary pairs and their use as calibration standards for various techniques used to measure the interaction of biological molecules. The nucleotide sequence of the nucleic acids are non-naturally occurring and the single-stranded nucleic acids are essentially lacking in secondary structure, that is, the stability of hybridization between a single stranded nucleic acid and its complement is greater than any intra-strand pair binding. Characteristics of a binding pair can be used to standardize data across experiments, across instruments and across platforms.

### CROSS REFERENCE TO RELATED APPLICATIONS OF THE INVENTION

This application is a Continuation-In-Part of PCT/US2014/030416 filed on Mar. 17, 2014 and Published as WO 2014/145621 on Sep. 18, 2014. This application also claims priority to U.S. Provisional Application No. 61/798,409 filed on Mar. 15, 2013. The contents of these applications are incorporated by reference in their entirety into the present application.

### STATEMENT OF RIGHTS UNDER FEDERALLY-SPONSORED RESEARCH

This invention was made with government support under grant number GM023037 awarded by the National Institutes of Health and grant number MCB1101859 awarded by the National Science Foundation. The government has cetain rights in the invention.

### SEQUENCE LISTING

This application contains a Sequence Listing, created on Mar. 17, 2014; the file, in ASCII format, is designated 0794143A_SequenceListing_ST25.txt and is 3.6 kilobytes in size. The sequence listing file is hereby incorporated by reference in its entirety into the application.

### FIELD OF THE INVENTION

The invention relates to calibration standards for standardizing the use of analytical instrumentation and in particular nucleic acids that function as calibration standards across a range of techniques and instruments.

### BACKGROUND OF THE INVENTION

Characterizing interactions between and within biomolecules can be critical to understanding biology and to devising pharmaceuticals to intervene when normal biological pathways fail. A small arsenal of techniques is available to measure various aspects of biomolecular interactions, which can be generally classified into four categories: spectroscopic, calorimetric, delivery and imaging, and separation techniques. Spectroscopic techniques include Surface Plasmon Resonance (SPR), Nuclear Magnetic Resonance (NMR), Mass Spectrometry (MS), UV-Vis spectroscopy, and fluorescent and chemiluminescent techniques; calorimetric techniques include Isothermal Titration calorimetry (ITC) and Differential Scanning calorimetry (DSC); Separation techniques include gel electrophoresis, Liquid Chromatography, and Scintillation Proximity Assay (SPA). Delivery and imaging techniques that include the use of the herein sequences (or derivatives) as novel nucleic acid tags, links, tethers or inserts for transporting probes, peptides or proteins, other nucleic acids and therapeutics and thus, are not likely to bind to an endogenous nucleic acid or protein, but to which probes could be engineered. Considering the wide range of techniques, it is perhaps unsurprising that there can be significant difficulty in comparing results across platforms.

While many instruments use internal calibration standards (e.g. acid-base titrations in ITC), these standards are often meaningless in the context of other techniques.

For example, isothermal titration calorimetry (ITC) is a fast and robust method to study the physical basis of molecular interactions. The complementary calorimetry method, differential scanning calorimetry (DSC), also yields important information about thermal stability. A single well-designed experiment can provide complete thermodynamic characterization of a binding reaction, including Kd, ΔG, ΔH, ΔS and reaction stoichiometry (n). Modern calorimeters are sensitive enough to probe even weak biological interactions with a sensitivity of ±2 ncals making ITC and DSC very popular methods among biochemists, and indispensable to pharmacological chemists in the design and characterization of drugs. Although ITC and DSC have been applied to protein studies for many years, particularly in drug design, the application in the RNA field for fundamental research and application to RNA-based drug development is still immature. One formidable problem to a more common use of these powerful instruments is lack of a readily available, inexpensive, dependable standard RNA and protocols.

ITC has been applied in protein fields for many years. Similarly, DSC has been used in the study of proteins and DNA. Thus, there are many applications in the literature for one to learn and to apply ITC and DSC to proteins and DNA.

Until recently the instruments used for ITC and DSC were calibrated and standardized with pancreatic ribonuclease A. Instruments calibrated with this enzyme are useless for the study of RNA because this RNase is impossible to destroy without compromising the integrity of the sample chamber of the ITC or DSC. Thus, there is no solution for standardizing ITC and/or DSC for RNA studies. However, there has been a recent move to use ITC and DSC for RNA studies. The solution of having readily available materials and protocols for RNA provides a good standardization to study RNA interactions using ITC and DSC. The series of ITC and DSC running conditions including temperature, buffer salt concentration, sample concentrations and pH all add flexibility for studies not achievable with other technologies. However, the multitude of possibilities and combinations are a hindrance to initiating an investigation of fundamental RNA biochemistry or an RNA-based drug development.

The provision of protocols for RNA study with standard materials would allow the ITC and DSC user the ability to fully explore the versatility of the instruments for their investigatory purposes for RNA. A conventional format results in which one can study RNA interactions. The standardized protocol enables an easy transfer of analysis conditions in systematic steps to study one's own RNA molecules. In academic and corporate labs alike, there many times there is a lack of ‘experimental memory’ that is more difficult to pass on when standard protocols for instrumentation are not available. Certainly in academic labs, instruction in the use of such instruments in research is clearly difficult without standard tools and protocols.

As the use of ITC and DSC for the study of RNA interactions expands, the need for the standardization between instruments and protocols increases. Furthermore, it would be useful to have a well-characterized cross-platform biological reference standard.

### SUMMARY OF THE INVENTION

To this end, nucleic acid standards are disclosed as a well-characterized cross-platform biological reference standard. Nucleic acid standards provide an ideal cross-platform reference for several reasons: 1) nucleic acid base pairing interactions are “tunable” based on the base content and length; 2) oligonucleotides are readily available at low cost and high purity 3) oligonucleotides can be readily modified with a wide array of labels; and 4) they can be stored and handled without degradation.

Accordingly, the invention relates to biological calibration standards based on complementary DNA and RNA oligonucleotides that can be used as references for many experimental systems including ITC and DSC. Complementary 11-mer oligonucleotides, as well as truncated versions consisting of 7 nucleotides were designed in both DNA and RNA, the nucleic acids containing negligible secondary structure. In some embodiments, nucleic acids containing tandem repeats of either the 11-mer or 7-mer, are encompassed by the invention so long as the design is such that the single-strand nucleic acids only base pair with each other and do not self-base pair. To date a genomic search has not identified an identical duplex.

A standardized protocol and standards for calibration of ITC and DSC instruments used in analysis of RNA:RNA, RNA:protein and RNA:small molecule interaction are also provided.

In one aspect, the invention relates to a nucleic acid consisting of an eleven (11) nucleotide RNA sequence: 5′-GACGUGCGAAG-3′ (SEQ ID NO: 1) and its complement: 5′-CUUCGCACGUC-3′ (SEQ ID NO: 2).

In another aspect, the invention relates to an nucleic acid consisting of an eleven (11) nucleotide DNA sequence: 5′-GACGTGCGAAG-3′ (SEQ ID NO: 3) and its complement: 5′-CTTCGCACGTC -3′ (SEQ ID NO: 4).

In yet another aspect, the invention relates to a nucleic acid consisting of a seven (7) nucleotide RNA sequence: 5′-CGUGCGA -3′ and its complement: 5′-UCGCACG -3′.

In another aspect, the invention relates to an nucleic acid consisting of a seven (7) nucleotide DNA sequence: 5′-CGTGCGA -3′ and its complement: 5′-TCGCACG -3′.

In yet another aspect, the invention relates to a nucleic acid comprising one or more, tandem repeats of any one of SEQ ID NOS: 1-4 or the 7-mers listed above.

In one aspect, therefore, the invention relates to a nucleic acid consisting of the nucleotide sequence of: 5′-GACGUGCGAAGGACGUGCGAAG -3′ (SEQ ID NO: 5); 5′-GACGTGCGAAGGACGTGCGAAG -3′ (SEQ ID NO: 7); 5′-CGUGCGACGUGCGA -3′ (SEQ ID NO: 9); 5′-CGUGCGACGUGCGACGUGCGA -3′ (SEQ ID NO: 10); 5′-CGTGCGACGTGCGA -3′ (SEQ ID NO: 13); 5′-CGTGCGACGTGCGACGTGCGA -3′ (SEQ ID NO: 14) and complements thereof.

One of skill will recognize that the nucleotide sequences can be modified by the addition or deletion of a nucleotide or two as long as the resulting nucleic acid contains negligible secondary structure but is still able to achieve the formation of a duplex between the nucleic acid and its complement. As stated above, nucleic acids of the invention encompass those in which the stability of hybridization between a single stranded nucleic acid and its complement is greater than any intra-strand pair binding.

In another aspect, therefore, the invention relates to a synthetic nucleic acid comprising from 5-22 and in some embodiments from 7 to 15 nucleotides and which has essentially no secondary structure as determined by as determined by a predictive modeling program (MFold). In some embodiments, the nucleic acid has essentially no secondary structure below 14 nucleotides and no substantial secondary structure of worthy stability (as described above) beyond 15 nucleotides as determined by a predictive modeling program (MFold.) The invention also includes 6-, 7-, 8-, 9-and 10-mers derived from the 11 nucleotide sequence.

In another aspect, the invention relates to the use of complementary nucleic acid pairs as calibration standards for various techniques used to measure the interaction of biological molecules; one such technique is isothermal titration calorimetry (ITC). A method for ITC using the oligonucleotides of the invention as calibration standards comprises the steps of (a) determining a set of parameters for an ITC instrument or experiment; (b) titrating a first solution (titrate solution) comprising a nucleic acid consisting of the nucleotide sequence of any of claims 1-4 into a second solution (titrant solution) comprising a nucleic acid that is the complement of the nucleic acid in said first solution (titrate) in said instrument or experiment; (c) measuring heat of reaction for reaction of titrate and titrant; wherein the heat of reaction determined for said titrate/titrant is characteristic of said set of parameters in said ITC instrument or experiment.

The calibration standards and protocol described herein are useful for standardization of, inter alia, ITC/DSC results between instruments and between experiments.

### BRIEF DESCRIPTION OF THE DRAWINGS

FIGS. 1A-C show isothermal titration calorimetry of the RNA 11-mer duplex at 37° C. A) Representative raw data of the power required to maintain a constant temperature (exotherm up) for 2.45 μL injections of 18.9 μM RNA 11-mer strand “B” into 2.3 μM of RNA 11-mer strand “A” at 37° C. B) Total heat of each injection (squares), determined by integrating the area under each of the curves from (A) for the first 150 seconds following injection with a 2 state fit of the data overlaid (solid line). C) Summary of thermodynamic data from triplicate experiments for all 8 conditions.

FIGS. 2A and B show differential scanning calorimetry of nucleic acid duplexes. A) heating and cooling curves for the RNA 11 base pair duplex show minimal hysteresis (˜0.5° C.) at 2° C./min, and near identical peak areas. B) Representative heating traces for all 4 duplexes with arbitrary y-offsets. Gray lines show baselines used for determining integrated area to get AH.

FIG. 3A-D shows UV-vis spectrophotometry of the 11 base pair RNA duplex melting. A) Representative raw traces of Absorbance as a function of temperature for heating and cooling ramps of 1 C/min. Small divergence between curves occurs at higher temperatures as evaporation increases the concentration slightly. B) Normalized melt curves at 6 different concentrations show the increase in melting temperature with concentration. C) Van't Hoff plot of curves from panel B, D) Linear plot showing concentration dependence of the melting temperature.

FIG. 4 shows absorbance vs. temperature for individual strands.

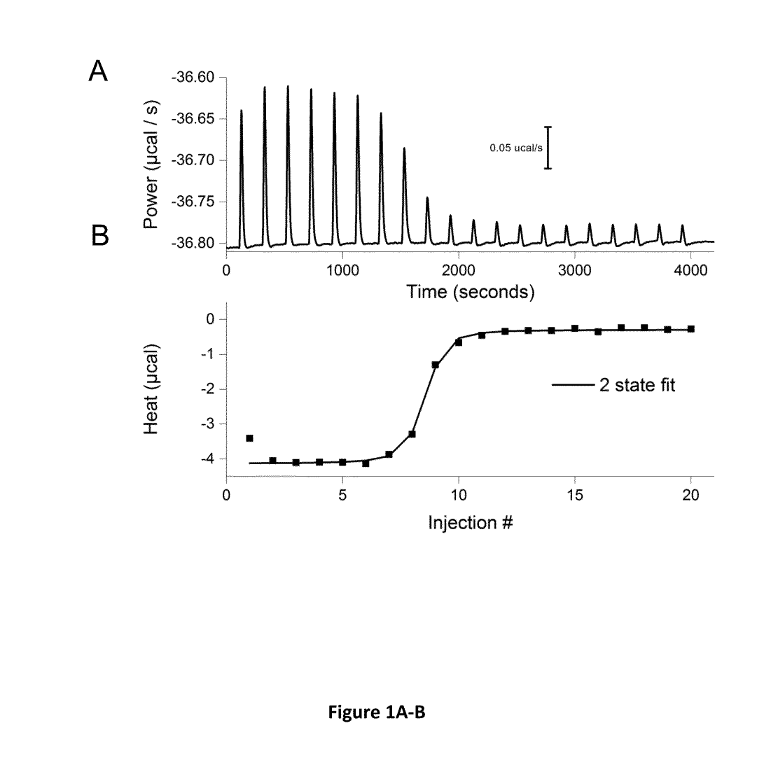

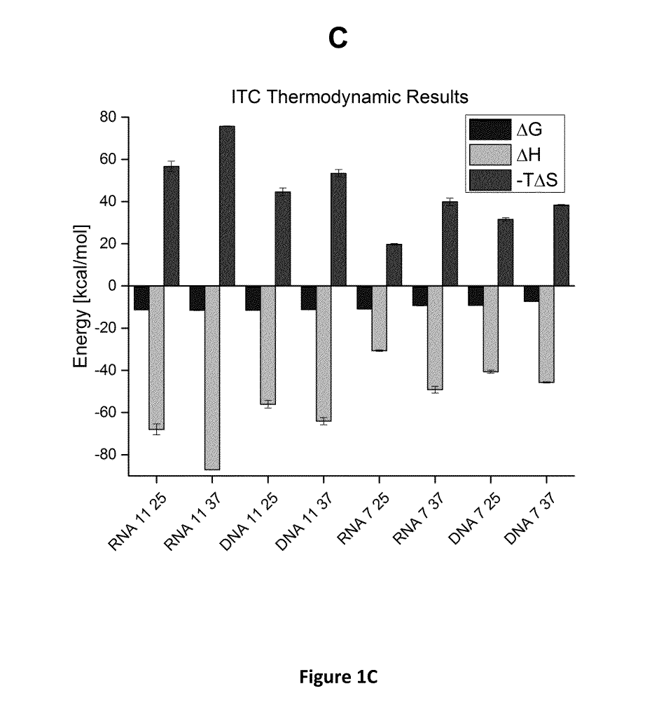

### DETAILED DESCRIPTION OF THE INVENTION

All patents, publications, applications and other references cited herein are hereby incorporated by reference into the present application.

Methodology used in developing the present invention are well known to those of skill in the art and are described, for example, in Oligonucleotide Synthesis, 1984 (M. L. Gait ed.), and The UNAFoId Web Server (url: mfold.rit.albany.edu). The contents of these references are hereby incorporated by reference in their entire into the present disclosure.

### Identification of Nucleic Acids

The present disclosure relates to nucleic acids, either RNA or DNA, and in some embodiments, a pair or set of complementary RNA or DNA oligos to be used as calibration standards for biomolecular interaction measurements. A nucleic acid of the invention can be from 7-15 nucleotides, or in some embodiments, from 5-22 nucleotides, in length as long as it exhibits no secondary structure and is capable of binding its corresponding complementary sequence (“complement”) to form a nucleic acid duplex, full or partial complement, the latter with one or two uncomplemented (“hanging or sticky”) ends. Methods for the synthesis of nucleic acids are well known in the art. Tools for predicting the secondary structure of an RNA or DNA, mainly by using thermodynamic methods, are also well known in the art and more detailed information regarding these tools can be found at the UNAFoId Web Server (url: mfold.rit.albany.edu).

An initial complementary pair of 11-nucleotide RNA nucleic acids was identified. Predictions in MFold showed that these sequences exhibited no secondary structure, and a BLAST search revealed no corresponding genomic sequence.

| | | |
|---|---|---|
| RNA1: | 5′-GACGUGCGAAG-3′ | (SEQ ID NO: 1) |
| RNA2: | 5′-CUUCGCACGUC-3′ | (SEQ ID NO: 2) |

The two complementary strands (RNA1 and RNA2) were obtained from Dharmacon (Thermo Scientific). The 2′-ACE protecting group was removed using a pH 3.8 deprotection buffer (supplied by Dharmacon). The samples were dialyzed extensively against water overnight before preliminary experiments were commenced. Initial studies indicated a strong exothermic event. Non [symbol] linear least square fit using a “one-site binding” model gave Ka=7 nM, n=0.99 (stoichiometric binding) and ΔH=-55.1 kcal/mole.

### Isothermal Titration Calorimetry (ITC) of RNA1 and RNA2

Experimental parameters to be measured include but are not limited to cell temperature, number of injections, injection volume, injection spacing, titrate/titrant concentration, etc. (1) In some embodiments, various conditions such as RNA concentration, pH, temperature and salts for the interaction of the two RNAs were optimized by both a Microcal ITC (GE Life Sciences) and Nano ITC (Waters).

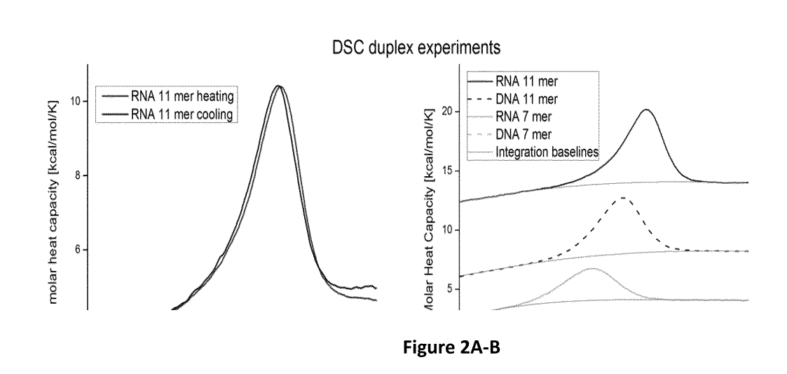

In one experiment, 100 μM RNA1 was titrated into 10 μM RNA2 in 10 mM phosphate buffer (pH7.0) at 25° C. The ITC profile is shown in FIG. 2, which indicates a strong exothermic event. Non-linear least square fit using “one-site binding” model shown in black solid line gave No Mg2+ is required for the RNA1 and RNA2 interaction. A similar binding constant was obtained when switching the RNA1 and RNA2 titration.

Other sequences identified include 7-and 11-nucleotide sequences of DNA or RNA as well as tandem repeats of the 7-mer and 11-mer sequences. Some embodiments of the 7-mer and 11-mer sequences of the invention are shown in Table 1. Some embodiments of nucleic acids with sequences of tandem repeats are shown in Table 2.

TABLE 1

| SEQ | | | | |
|---|---|---|---|---|
| ID | | | MW | Extinction |
| NO | Designation | Sequence | (Da.) | Coefficient* |
| 1 | R11A | GACGUGCGAAG | 3568.2 | 98529 |
| 2 | R11B | CUUCGCACGUC | 3402.1 | 88041 |
| 3 | D11A | GACGTGCGAAG | 3406.3 | 99081 |
| 4 | D11B | CTTCGCACGTC | 3268.2 | 84475 |
| 5 | R7A | CGUGCGA | 2219.4 | 58056 |
| 6 | R7B | UCGCACG | 2179.4 | 58251 |
| 7 | D7A | CGTGCGA | 2121.4 | 60685 |
| 8 | D7B | TCGCACG | 2081.4 | 57095 |

*Measured values at A260~0.2

TABLE 2

| | | Basic | |
|---|---|---|---|
| SEQ | | Sequence | |
| ID | | (No. of | No. of |
| NO | Sequence | Nucleotides) | Repeats |
| 1 | GACGUGCGAAG | 11 | 1 |
| 2 | CUUCGCACGUC | 11 | 1 |
| 3 | GACGTGCGAAG | 11 | 1 |
| 4 | CTTCGCACGTC | 11 | 1 |
| — | CGUGCGA | 7 | 1 |
| — | UCGCACG | 7 | 1 |
| — | CGTGCGA | 7 | 1 |
| — | TCGCACG | 7 | 1 |
| 5 | GACGUGCGAAGGACGUGCGAAG | 22 | 2 |
| 6 | CUUCGCACGUCCUUCGCACGUC | 22 | 2 |
| 7 | GACGTGCGAAGGACGTGCGAAG | 22 | 2 |
| 8 | CTTCGCACGTCCTTCGCACGTC | 22 | 2 |
| 9 | CGUGCGACGUGCGA | 14 | 2 |
| 10 | CGUGCGACGUGCGACGUGCGA | 21 | 3 |
| 11 | UCGCACGUCGCACG | 14 | 2 |
| 12 | UCGCACGUCGCACGUCGCACG | 21 | 3 |
| 13 | CGTGCGACGTGCGA | 14 | 2 |
| 14 | CGTGCGACGTGCGACGTGCGA | 21 | 3 |
| 15 | TCGCACGTCGCACG | 14 | 2 |
| 16 | TCGCACGTCGCACGTCGCACG | 21 | 3 |

First, the room temperature extinction coefficients of the oligos were measured at 260 nm to ensure accurate determination of the concentrations that are requisite for extracting thermodynamic parameters. DNA and RNA oligos were fully hydrolyzed using phosphodiesterase I from snake venom and room temperature extinction coefficients were calculated using base extinction coefficients and the ratio of intact vs. hydrolyzed oligos^{1,2}. The values are shown in Table 1 (above), and the errors are estimated to be less than 2% based on the primary source of measurement error—concentration changes due to evaporation from the cuvettes. Consistent with previous work¹, the measured values were significantly lower than calculated extinction coefficients for these sequences. For example, the manufacturer (Integrated DNA technologies) reported extinction coefficients for the RNA 11-mers of 113000 L/mol-cm and 96700 L/mol-cm for strands A and B, overestimating the measured values by 11% and 10%, respectively. Additionally, the change in absorbance of the individual strands was measured for a temperature ramp between 20° C. and 90° C. (FIG. 7). The RNA “A” strands have changes in absorbance near room temperature but none of the other strands show this behavior. This behavior is attributed to a tendency of the “A” strands to form homodimers, which is supported by predictions in Mfold and supported by other experimental evidence from ITC data at different temperatures (see below). It is likely not seen in the DNA “A” strands due to the lower stability of the homodimers in DNA than in RNA. The changes in absorbance for the individual strands during temperature ramping do not recapitulate the changes seen from the digestion. This suggests that high temperature should not be substituted for digestion in determining accurate extinction coefficients for oligos, as is sometimes done^{9,10}.

One of the most common and useful measures of biomolecular interactions is the association constant Kₐ (or its inverse K_{D}), which reports the affinity of the two molecules at equilibrium. Isothermal Titration calorimetry (ITC) was used to measure this as well as the thermodynamics of the interactions. Using ITC, the exothermic heat of reacting one strand with the other was measured as the duplex was formed. A representative experiment is shown in FIG. 2a, with the resultant data and 2-state model fit in FIG. 2b. Experiments were performed in triplicate from a single set of working solutions whose concentrations were determined by a Nanodrop spectrophotometer using the extinction coefficients previously determined. The data from experiments in PBS at 25° C. and 37° C. is summarized in Table 3 and FIG. 2c.

TABLE 3

| | RNA 11 mer | | DNA 11 mer | | RNA 7 mer | | DNA 7 mer | |
|---|---|---|---|---|---|---|---|---|
| Temperature (° C.) | 25 | 37 | 25 | 37 | 25 | 37 | 25 | 37 |
| [A strand] (μM)* | 2.3 | | 3.1 | | 6.7 | 6.9 | 7.6 | 51.6 |
| [B strand] (μM)* | 18.2 | | 20.1 | | 34.9 | | 52.4 | 221.4 |
| Stoichiometry | 1.02 ± .05 | 0.99 ± .01 | 1.03 ± .01 | 1.01 ± .02 | 0.98 ± .02 | 0.94 ± .02 | 1.07 ± .02 | 0.90 ± .01 |
| Ka (1/μM) | 205 ± 30 | 125 ± 41 | 268 ± 41 | 77 ± 20 | 102 ± 12 | 3.6 ± 0.8 | 5.0 ± 0.3 | 0.16 ± .01 |
| ΔH (kcal/mol) | −68.0 ± 2.6 | −87.1 ± 0.9 | −56.1 ± 1.8 | −64.1 ± 1.7 | −30.7 ± 0.3 | −49.2 ± 1.6 | −40.7 ± 0.7 | −45.7 ± 0.3 |
| ΔS (cal/mol/K)^{$} | −190.0 ± 8.4 | −244.0 ± 0.5 | −149.5 ± 6.4 | −170.6 ± 5.3 | −66.2 ± 1.3 | −128.5 ± 5.6 | −105.8 ± 2.5 | −123.6 ± 0.8 |
| ΔG (kcal/mol)^{$} | −11.3 ± 0.1 | −11.5 ± 0.2 | −11.5 ± 0.1 | −11.2 ± 0.2 | −10.9 ± 0.1 | −9.3 ± 0.1 | −9.14 ± 0.04 | −7.38 ± 0.05 |

Note:
errors represent only statistical variations between experiments
*Merged cells signify a common solution for multiple experiments
^{$}Calculated values from other parameters

As expected, stoichiometries for all experiments were measured near 1 and typically within a few percent of 1, indicating a 1:1 reaction between strands. For all duplexes, the stoichiometry decreased as the temperature was increased from 25° C. to 37° C., even when the same stock solutions were used for both temperatures. This suggests that at lower temperatures, some fraction of strands is unavailable to form duplex during the injection due to self-interaction or dimerization. This deviation was especially dramatic for the shorter strands, which is likely due to higher uncertainty in concentration measurement at the higher concentrations used due to homodimer formation. It could also be partially attributed to the greater difficulty in accurately determining the stoichiometry as the association constant decreases.

At 37° C., where self-interactions and homodimers are minimized, association constants and enthalpies behaved as expected, increasing with longer strands and increasing from DNA to RNA. Somewhat counter intuitively, the enthalpy of formation of all 4 duplexes becomes more favorable as the temperature increases from 25° C. to 37° C. This again suggests that the oligos have some structure at 25° C., but that much of this structure is removed once the temperature is increased to 37° C. This is consistent with the tendency of the “A” strands to form homodimers. The ITC data suggests that nearly all of the material is forming duplex at these conditions (within 150 seconds), but that there is a small enthalpic penalty for removing structure from the isolated oligo solutions before forming the enthalpically favorable duplex.

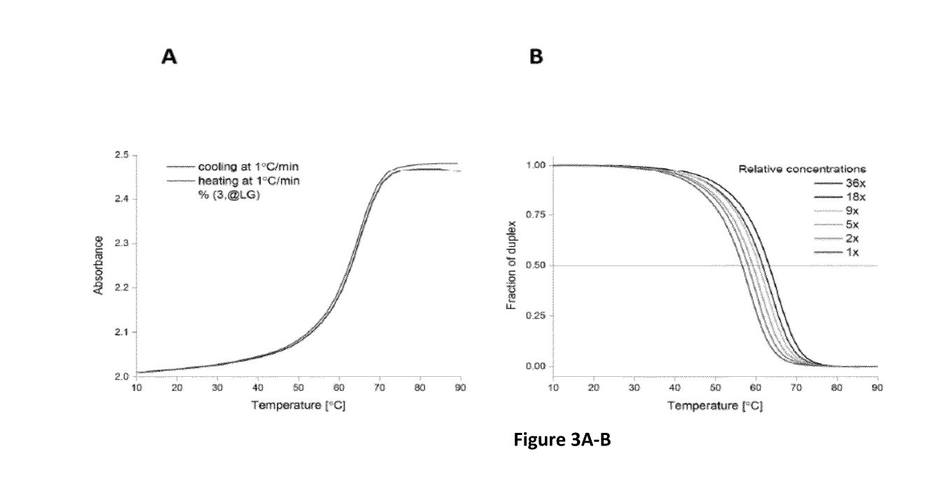

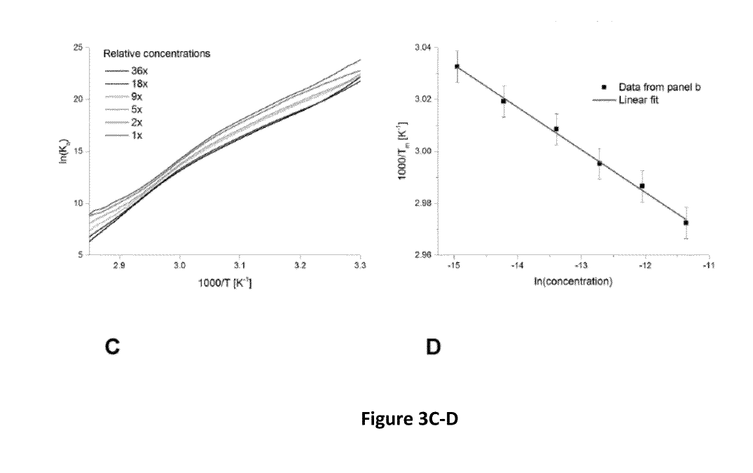

Complementary experiments were performed by heat denaturing the duplexes using temperature ramps. These were performed using both DSC, which measures the power required to keep a sample cell and reference cell at the same temperature, and UV melting, which measures change in absorbance due to base stacking. The DSC experiments were performed by ramping the temperature from 10° C. and 100° C. at 2° C./minute. The pressure was held at 3 atmospheres, enough to prevent evaporation but not to significantly affect thermodynamics of duplex formation 11. Data between heating and cooling cycles was repeatable, showing only a fraction of a degree shift in melting temperature in the most extreme cases (FIG. 3a). FIG. 3b shows the molar heat capacity for all 4 duplexes, where the integral between the curve and baseline is equal to ΔH between the folded and unfolded states (data shown in table 3). These data follow the same trend as ITC data, and for 3 of the 4 duplexes the enthalpies are within about 10% of those determined from ITC at 37° C. The errors listed from the table are statistical errors from the automated integration routine, but we noticed that choice of baseline endpoints and shape (e.g. linear, cubic, spline, etc.) could result in changes up to 10% as well. The 7 mer DNA differs substantially from ITC experiments, underestimating ΔH by almost 40%. We believe this is likely due to the difficulty in establishing a reliable baseline since this duplex shows a steep pre-transition phase and a low melting temperature. The entropy values were determined by dividing the curve by absolute temperature and integrating the area between the curve using the same spline parameters. We found entropies that were within error of RNA values calculated from ITC values at 37° C., and within 30% for those of DNA.

It is worth noting that the curves all exhibit some asymmetry with a more gradual rise at low temperatures than the fall at high temperatures. This behavior is most pronounced for the 11 mers, and for the RNA in particular, suggesting less than perfect two-state behavior.

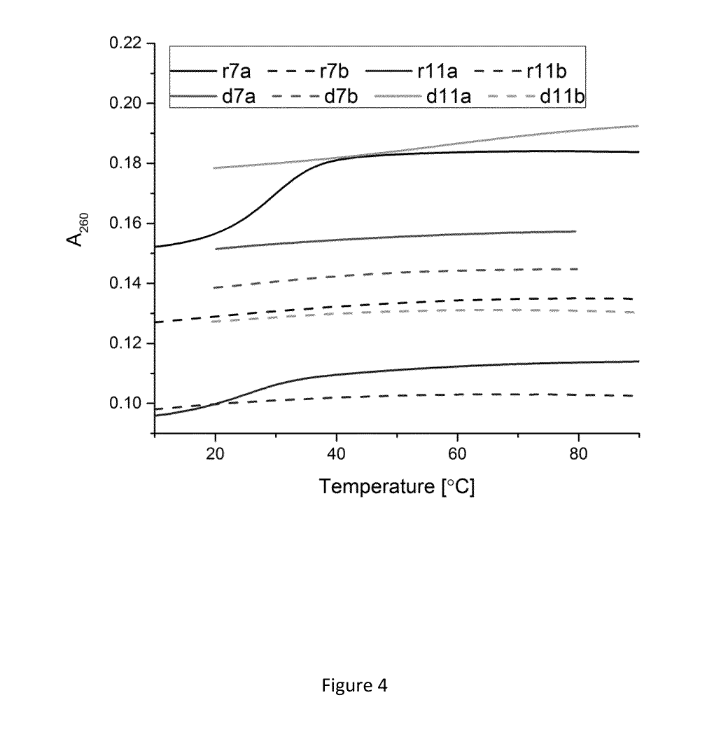

For UV melt experiments, similar experiments were performed, where the 260nm absorbance of duplexes measured the change in base stacking. Again we found repeatable data between subsequent heating and cooling cycles, with only slight shifts in absorbance at high temperature due to evaporation (FIG. 4A). We measured the melting behavior at a variety of different concentrations, and found increasing melting temperatures as concentrations were increased (FIG. 4B). Enthalpies of formation were determined from the data by two methods: van't Hoff analysis (FIG. 4C) and concentration dependence of melting temperature (FIG. 4D). The data is summarized in Table 3 (above). Van't Hoff analysis shows some non-linearity for all duplexes, while the concentration dependence of the melting temperature shows near perfect linearity over the concentrations probed. Enthalpies determined from these methods were typically more negative than those determined from the other methods, sometimes dramatically so. This likely arises since both analysis methods are valid only for two state reactions, and we have already shown deviation from that behavior in the previous experiments.

### EXAMPLES

### Materials

Oligonucleotides were purchased from IDT with standard desalting and deprotection. IDT claims an average coupling efficiency of 99.2% per reaction, giving an approximate full length yield of 92% for 11-mers and ˜95% for 7-mers. We verified the purity of the oligos using UPLC (Waters Corporation) and found a dominant single peak consistent with published purities. PBS buffer was made from a 10× concentrated solution (Fisher Scientific BP399-500), with the 1× solution consisting of 11.9 mM Phosphates, 137 mM Sodium Chloride, and 2.7 mM Potassium Chloride at pH 7.4.

### Extinction Coefficient Measurements

Extinction coefficients for the 8 oligos were determined by monitoring UV absorbance of individual oligo solutions during digestion by Phosphodiesterase I as described previouslyl. Disposable semi-micro UV transparent cuvettes (Brand Scientific) were filled with 1.6 mL of PBS and individually zeroed. Concentrated oligos were added to each cuvette except for one blank (20 μL for DNA and 30 μL for RNA—to give an absorbance between 0.1 and 0.25) and mixed before measuring the absorbances again. 10 μL of Phosphodiesterase I from snake venom (Sigma) was added to each cuvette. The extinction coefficient at room temperature was calculated by applying the sum of nucleotide absorbances to the hydrolyzed oligos, and extrapolating to the room temperature absorbancest^{1,2}.

### Isothermal Calorimetry (ITC)

All ITC experiments were performed using the low volume Nano ITC (TA Instruments). The instrument was factory calibrated using KHCO3-HCL titrations to determine the effective cell volume. The injection syringe was calibrated by mass of water 3,4. Experiments consisted of a solution of one oligo (the “B” strand) was titrated into a solution of its complement in the cell (the “A” strand) using twenty 2.5 μL injections spaced 200 s apart, with 350 rpm stirring. The buret was driven to 99% volume before the syringe was loaded to minimize the effects of mechanical backlash5. Experiments were performed in triplicate at various temperatures, using the same stock solutions where possible. The concentrations of the various strands for each experiment are reported in Table 2. Integration of the heat curves was performed using the NanoAnalyze software (TA Instruments), with automated baseline construction, integrating from the injection start to 150s post injection. The curves were individually fit with 2 state binding models with a constant baseline adjustment to account for the mixing enthalpy contribution. This constant baseline was taken as a single value for all runs at a given temperature, determined by averaging the fit result from 6 runs comprising the DNA and RNA 11-mers, which gave well-defined baselines. The first data point was excluded from analysis as is the convention. Additionally, there was one other injection anomaly (representing 0.2% of the data), which was ignored in fitting but treated as a partial injection in determining the stoichiometry. Final values and error estimates for enthalpy, stoichiometry, and Ka were determined by using the mean and standard deviation of the fitted results for each of the triplicate experiments.

Measurements of duplex formation by Isothermal Titration calorimetry are Shown in Table 3 (above).

### UV-Vis Experiments

All UV melting experiments were performed in an Agilent Cary 100 system. Semi-micro quartz cuvettes held 1 mL DNA or RNA solution at various concentrations. The temperature was ramped between 10° C. and 90° C. at 2° C./min for duplex melting experiments, and these conditions varied slightly for individual strand melting experiments (melting temperatures were unaffected by changing ramp speeds from 1° C./min to 2° C./min). The temperature was monitored in a dummy cuvette at the height of the light path. Experiments were performed in triplicate and curves were averaged (decide on averaging just heating curves or both). Normalizing raw data traces by the high temperature absorbance collapsed all three curves together, suggesting that variation between curves is mostly due to evaporation. Forward and reverse curves were also superimposable in this way, suggesting that our ramp rate was slow enough to allow for equilibration at all temperatures. Averaged curves were converted to curves of fraction folded as described elsewhere⁶. Regions of full association and dissociation were chosen differently for each of the 4 duplexes, but the same for various concentrations of each duplex. Two types of melting analysis were performed: 1) Van't Hoff analysis of individual melt curves, and 2) analysis of the concentration dependence of the melting temperature.

Measurement of duplex formation by DSC and UV melts is shown in Table 4.

TABLE 4

| | RNA 11 mer | DNA 11 mer | RNA 7 mer | DNA 7 mer |
|---|---|---|---|---|
| ΔH_{DSC} | −81.9 ± 2.6 | −70.9 ± 1.3 | −45.4 ± 3.0 | −28.7 ± 2.0 |
| (kcal/mol) | | | | |
| ΔH_{VH} | −82.7 | −78.9 | −63.4 | −52.1 |
| (kcal/mol) | | | | |
| ΔH_{Conc} | −121.6 | −86.1 | −74.4 | −70.3 |
| (kcal/mol) | | | | |
| ΔS_{DSC} | −240 ± 10 | −212 ± 9 | −139 ± 14 | −87 ± 9 |
| (cal/mol/K) | | | | |
| ΔS_{VH} | TBD | TBD | TBD | TBD |
| (cal/mol/K) | | | | |
| ΔS_{Conc} | TBD | TBD | TBD | TBD |
| (cal/mol/K) | | | | |

### Differential Scanning Calorimetry (DSC)

All DSC experiments were performed on a Nano DSC (TA Instruments) with a 300 μL cell volume. Duplexes were formed by mixing the complementary DNA or RNA strands in equimolar concentrations from various stock concentrations (between 95 and 142 μM). The duplexes were dialyzed against 500 mL of PBS buffer for 18 hours at 4° C. (Sigma PURD10005 with 1 kDa cutoff) to ensure matching conditions between sample and reference cells. The 260 nm absorbances of the dialyzed duplexes were measured before the experiment. Final duplex concentrations were as follows: 47.6 μM for RNA 11-mer, 63.1 μM for DNA 11-mer, 65.5 μM for RNA-7mer, and 65.8 μM for DNA 7-mer. Both the sample and matching reference buffer were extensively degassed under vacuum for at least 20 minutes to prevent bubbles. Scans were performed from 10° C. to 100° C. at 2° C./min, at a pressure of 3 atmospheres. The first heating and cooling cycle of each experiment was discarded due to its unique thermal history. Three additional heating and cooling scans were performed to ensure reproducibility. A reference scan was made under the same conditions using reference buffer in both cells. Data analysis was performed using the Nanoanalyze software (TA Instruments) and Origin 9.1 (Originlabs). Two representative reference scans (one each for heating and cooling) were subtracted from all duplex scans, and a partial specific volume for RNA was assumed to be 0.5 cm³/g based on previous reports⁷. We used spline interpolation between pre-transition and post-transition regions to define the baseline that was used for integration and determination of ΔH and ΔS⁸. Specifically, a spline was fit to 6 data points consisting of 3 low temperature points and 3 high temperature points. These temperatures were chosen consistently for each duplex, but varied slightly between the 7-and 11-mers to accommodate shifting melting temperatures. For each duplex, the three heating scans and three cooling scans were individually analyzed and the results were averaged.

### REFERENCES

1. Kallansrud, G. & Ward, B. A comparison of measured and calculated single-and double-stranded oligodeoxynucleotide extinction coefficients. Analytical Biochemistry 236, 134-138 (1996).

2. Cavaluzzi, M. J. Revised UV extinction coefficients for nucleoside-5′-monophosphates and unpaired DNA and RNA. Nucleic Acids Research 32, 13e-13 (2004).

3. Demarse, N. A., Quinn, C. F., Eggett, D. L., Russell, D. J. & Hansen, L. D. Analytical Biochemistry. Analytical Biochemistry 417, 247-255 (2011).

4. Tellinghuisen, J. Calibration in isothermal titration calorimetry: Heat and cell volume from heat of dilution of NaCI(aq). Analytical Biochemistry 360, 47-55 (2007).

5. Mizoue, L. S. & Tellinghuisen, J. The role of backlash in the ‘first injection anomaly’ in isothermal titration calorimetry. Analytical Biochemistry 326, 125-127 (2004).

6. Mergny, J.-L. & Lacroix, L. Analysis of thermal melting curves. Oligonucleotides 13, 515-537 (2003).

7. Durchschlag, H. in Thermodynamic data for biochemistry and biotechnology 45-128 (Springer, 1986).

8. Spink, C. H. in Methods in Cell Biology 84, 115-141 (Elsevier, 2008).

9. Pan, T. & Sosnick, T. R. Intermediates and kinetic traps in the folding of a large ribozyme revealed by circular dichroism and UV absorbance spectroscopies and catalytic activity. Nat. Struct. Biol. 4, 931-938 (1997).

10. Hartmann, R. K. R. K. Handbook of RNA biochemistry. (Weinheim : Wiley-VCH, 2009).

11. Macgregor, R. B. Chain length and oligonucleotide stability at high pressure. Biopolymers 38, 321-328 (1996).

12. Holbrook, J. A., Capp, M. W., Saecker, R. M. & Record, M. T. Enthalpy and Heat Capacity Changes for Formation of an Oligomeric DNA Duplex: Interpretation in Terms of Coupled Processes of Formation and Association of Single-Stranded Helices †. Biochemistry 38, 8409-8422 (1999).

13. Liu, Y. & Sturtevant, J. M. Significant discrepancies between van't Hoff and calorimetric enthalpies. III. Biophys. Chem. 64, 121-126 (1997). 14. Chaires, J. B. Possible origin of differences between van't Hoff and calorimetric enthalpy estimates. Biophys. Chem. 64, 15-23 (1997). 15. Horn, J. R., Russell, D., Lewis, E. A. & Murphy, K. P. Van't Hoff and calorimetric enthalpies from isothermal titration calorimetry: are there significant discrepancies? Biochemistry 40, 1774-1778 (2001).

16. Naghibi, H., Tamura, A. & Sturtevant, J. M. Significant discrepancies between van't Hoff and calorimetric enthalpies. P Natl Acad Sci Usa 92, 5597-5599 (1995).

17. Feig A L, Studying RNA-RNA and RNA-Protein Interactions by Isothermal Titration Calorimetry. Methods Enzymol. 2009; 468: 409-422

18. Salim N N and Feig A L, Isothermal Titration Calorimetry of RNA. Methods 2009 47(3): 198-205

1. A synthetic nucleic acid comprising from 5 to 22 nucleotides, said nucleic acid having essentially no secondary structure as determined by a predictive modeling program (MFold.)

2. The nucleic acid of claim 1 comprising from 7 -11 nucleotides.

3. The synthetic nucleic acid of claim, wherein the nucleic acid is RNA.

4. The synthetic nucleic acid of claim, wherein the nucleic acid is DNA.

5. The synthetic nucleic acid of claim 1 consisting of a nucleotide sequence selected from the group consisting of:
(SEQ ID NO: 1)
GACGUGCGAAG;
(SEQ ID NO: 2)
CUUCGCACGUC;
(SEQ ID NO: 3)
GACGTGCGAAG;
(SEQ ID NO: 4)
CTTCGCACGTC;
(SEQ ID NO: 5)
CGUGCGA; UCGCACG; CGTGCGA; TCGCACG;
GACGUGCGAAGGACGUGCGAAG;
(SEQ ID NO: 6)
CUUCGCACGUCCUUCGCACGUC;
(SEQ ID NO: 7)
GACGTGCGAAGGACGTGCGAAG;
(SEEQ ID NO: 8)
CTTCGCACGTCCTTCGCACGTC;
(SEQ ID NO: 9)
CGUGCGACGUGCGA;
(SEQ ID NO: 10)
CGUGCGACGUGCGACGUGCGA;
(SEQ ID NO: 11)
UCGCACGUCGCACG;
(SEQ ID NO: 12)
UCGCACGUCGCACGUCGCACG;
(SEQ ID NO: 13)
CGTGCGACGTGCGA;
(SEQ ID NO: 14)
CGTGCGACGTGCGACGTGCGA;
(SEQ ID NO: 15)
TCGCACGTCGCACG;
(SEQ ID NO: 16)
TCGCACGTCGCACGTCGCACG.

6. A pair of complementary synthetic nucleic acids, said pair comprising first and second synthetic nucleic acids, said first nucleic acid consisting of a nucleotide sequence selected from the group consisting of SEQ ID NO: 1, SEQ ID NO: 3, SEQ ID NO: 5, SEQ ID NO: 7, SEQ ID NO: 9, SEQ ID NO: 10, SEQ ID NO: 11, SEQ ID NO: 12, SEQ ID NO: 13, SEQ ID NO: 14, SEQ ID NO: 15; SEQ ID NO: 16; CGUGCGA; and CGTGCGA; and said second nucleic acid consisting of the nucleotide sequence complementary to the nucleotide sequence of said first nucleic acid.

7. A method for isothermal titration calorimetry comprising:
(a) determining a set of parameters for an ITC instrument or experiment;
(b) titrating a first solution comprising a nucleic acid consisting of the nucleotide sequence of claim 1 into a second solution comprising a nucleic acid that is the complement of the nucleic acid in said first solution in said instrument or experiment;
(c) measuring heat of reaction for reaction of said first solution (titrate solution) and said second solution (titrant solution);
wherein the heat of reaction determined for first solution (titrate solution)/second solution (titrant solution) is characteristic of said set of parameters in said ITC instrument or experiment.

8. The method of claim 6, wherein the nucleic acid in said first solution has the nucleotide sequence of SEQ ID NO: 1 and the nucleic acid of said second solution has the nucleotide sequence of SEQ ID NO: 2.

9. A kit consisting of one or more of the synthetic nucleic acids of claims 1-4 and its complementary sequence.

10. The synthetic nucleic acid of claim 1, wherein the nucleic acid is RNA.

11. The synthetic nucleic acid of claim 1, wherein the nucleic acid is DNA.
